(19) United States
(12) Patent Application Publication
(10) Pub. No.: US 2024/0172936 A1
(43) Pub. Date: May 30, 2024

(54) CYSTOSCOPY INSERTION TOOL

(71) Applicant: Crest Medical Ltd, Woolston, Warrington (GB)

(72) Inventor: James SEAL, Stowmarket, Suffolk (GB)

(21) Appl. No.: 18/280,934

(22) PCT Filed: Feb. 17, 2021
(86) PCT No.: PCT/GB2022/050432

§ 371 Date: Sep. 7, 2023

(30) Foreign Application Priority Data
Mar. 12, 2021 (GB) 2103481.4
Mar. 12, 2021 (GB) 2114782.2

(51) Int. Cl. A61B 1/307; A61B 1/00; A61B 10/04

(52) U.S. Cl. CPC A61B 1/307; A61B 1/00135; A61B 1/00154; A61B 10/04

(57) ABSTRACT

A cystoscopy insertion tool (100) for inserting a cystoscope into the body is described herein. The tool comprises an inner housing (103) having an aperture at a proximal end and an aperture at a distal end with a first lumen extending therebetween for receiving a cystoscope therethrough, and an outer housing (101) having an aperture (122) at a proximal end and an aperture (120) at a distal end having a longitudinal axis extending therebetween and providing a second lumen extending therebetween for receiving the inner housing such that the inner housing is at least partially nested inside the outer housing. The outer housing comprises a first port (108) and a second port (110), and wherein the first and second ports comprise respective conduits that extend from the outer housing at an angle relative to the longitudinal axis. A channel is formed between the inside of the outer housing and the outside of the inner housing when the inner housing is nested in the outer housing, to allow the passage of fluid and/or a biopsy probe to pass from the respective first and second ports and through the channel.

### FIELD OF THE INVENTION

The present disclosure relates to a tool for inserting a cystoscope into a body cavity, such as the bladder.

### BACKGROUND

A cystoscope is a device used to look inside a body cavity or the human or animal body, such as the bladder. In a cystoscopy procedure, the cystoscope is normally inserted through a natural opening, such as the urethra to gain access to the bladder. The cystoscope may be wrapped or protected in a sheath which may aid insertion. The cystoscope generally transmits an image from the inside of the body cavity along its shaft to the operator or a video screen via, for example, a fibre optic cable or bundle.

In some cystoscopy procedures, it can be advantageous to irrigate the site such as the bladder to clear away any debris and to expand the cavity to improve visibility. For example, in a cystoscopy procedure water or saline may be delivered into the bladder to fill the bladder. This stretches the bladder wall allowing the operator to view the entire bladder wall.

WO 2008/122785 describes an apparatus for deploying an endoscope. The apparatus comprises a means for temporarily applying fluid pressure to the inside of a sheath to expand the sheath and facilitate insertion of the instrument in the body cavity. Fluid may be fed via an inlet to the sheath. However, due to the shape and orientation of the inlet, turbulence may occur creating uncomfortable air bubbles. In addition, the arrangement of the inlet relative to the insertion tool makes it difficult for any other tools or instruments, such as a biopsy tool, to be fed to the site being imaged.

There therefore exists a need to provide an improved tool for cystoscopy that is more comfortable for the patient and that facilitates the use of a tool such as a biopsy tool at the same time as the site being imaged, such as the bladder, is irrigated.

### SUMMARY OF THE INVENTION

Aspects of the invention are as set out in the independent claims and optional features are set out in the dependent claims. Aspects of the invention may be provided in conjunction with each other and features of one aspect may be applied to other aspects.

In a first aspect there is provided an insertion tool for inserting an endoscope such as a cystoscope into the body. The tool may therefore be a cystoscopy insertion tool. The insertion tool comprises an inner housing having an aperture at a proximal end and an aperture at a distal end with a first lumen extending therebetween for receiving a cystoscope therethrough, and an outer housing having an aperture at a proximal end and an aperture at a distal end having a longitudinal axis extending therebetween and providing a second lumen extending therebetween for receiving the inner housing such that the inner housing is at least partially nested inside the outer housing, the outer housing comprising a first port and a second port, and wherein the first and second ports comprise respective conduits that extend from the outer housing at an angle relative to the longitudinal axis. A channel is provided between the inside of the outer housing and the outside of the inner housing when the inner housing is nested in the outer housing, to allow the passage of fluid and/or a biopsy probe to pass from the respective first and second ports and through the channel. At least a portion of the external profile of the inner housing is tapered, wherein the degree of tapering is based on the angle at which the respective conduits of the first and second ports of the outer housing extend at relative to the longitudinal axis of the outer housing.

By providing a tapered inner house where the degree of tapering is based on the angle at which at which the respective conduits of the first and second ports of the outer housing extend at relative to the longitudinal axis of the outer housing means that fluid and/or tools fed through the insertion tool to the site of inspection may be fed more easily and smoothly to the site of inspection. This may, for example, reduce turbulence in a fluid being delivered, thereby reducing the presence of uncomfortable air bubbles, but also means that a tool can be smoothly and easily operated by an operator.

In use, the inner housing may be nested within the outer housing and secured to the outer housing, for example via adhesive. Manufacturing an insertion tool in this way may make the manufacturing process easier and simpler, and also allow components to be made more accurately. The components may be made, for example, by injection moulding and by making the tool from two components in this manner may improve the tolerances to which each component is made and also reduce the likelihood that extraneous material may be formed and block the channel or one of the conduits.

The tapering of the external profile of the inner housing may be symmetrical about the longitudinal axis of the inner housing. In some examples, the tapering of the external profile of the inner housing may be at least partially arcuate. At least a portion of the external profile of the inner housing may be tapered along the length of the first region at a region proximate to the respective conduits of the first and second ports when the inner housing is nested inside the outer housing.

In some examples the steepest angle of the degree of tapering may match the angle at which the conduits of the outer housing extend at relative to the longitudinal axis of the outer housing. In examples where the tapering is arcuate, the angle of incline relative to the longitudinal may vary from a relatively steep angle (for example between 25 and 45 degrees, for example, between 30 and 35 degrees, for example 31 degrees) towards the proximal end of the inner housing matching the angle at which the conduits of the outer housing extend at relative to the longitudinal axis of the outer housing and gradually decrease along the longitudinal axis of the inner housing until the angle near distal end of the inner housing is much shallower (for example, between 1 and 5 degrees, for example 1 degree).

The tapering of the external diameter of the inner housing and the relative angle at which the respective conduits of the first and second ports of the outer housing extend at relative to the longitudinal axis of the outer housing may be selected to provide a laminar flow of fluid through the respective conduits and through the channel between the inside of the outer housing and the outside of the inner housing.

In some examples the outer housing is also tapered at least partially along its longitudinal axis. The tapering of the outer housing may match or mirror that of a corresponding tapered portion of the inner housing. In some examples the outer housing may have two regions of tapering, wherein the two regions have differing degrees of tapering. For example, a proximal end of the outer housing may have a greater or steeper degree of tapering (for example to aid at least partial insertion into a body cavity) whereas a more distal region of the outer housing may have a shallower degree of tapering.

The first and second ports may be provided on opposing sides of the outer housing such that when the outer housing is viewed in cross section along the longitudinal axis the first and second ports are opposite to each other.

The first and second ports each comprise a tab providing a female luer lock fitting.

The second lumen of the outer housing may be shaped relative to the dimensions of the inner housing to provide the channel between the inside of the outer housing and the outside of the inner housing.

The insertion tool may further comprise a first sheath coupled to the inner housing, wherein the first sheath is configured to enclose a cystoscope passing through the first lumen, and a second sheath coupled to the outer housing, the second sheath enclosing the first sheath and providing third and fourth lumens on opposing sides of the first sheath for receiving fluid and/or a biopsy tool from the first and second ports respectively via the channel between the inside of the outer housing and the outside of the inner housing.

The second sheath may have an oval cross-section when the cross-section is taken when viewed along the longitudinal axis, wherein each end of the oval provides the respective third and fourth lumens relative to the first sheath which is central to the oval.

A portion of the outer profile of the inner housing may comprise a lipped portion of reduced profile proximate to the distal end of the inner housing for receiving the first sheath. A portion of the outer profile of the outer housing may also comprises a lipped portion of reduced profile proximate to the distal end of the outer housing for receiving the second sheath.

The acute angle at which the respective conduits of the first and second ports of the outer housing extend at relative to the longitudinal axis of the outer housing may be in the range of 25 to 45 degrees, preferably 30 to 35 degrees, preferably 31 degrees.

In some examples the insertion tool further comprises a bung having an aperture, the bung configured to mate with the proximal end of the first lumen of the inner housing, the bung configured to receive and sealingly engage with a cystoscope inserted through the aperture and into the first lumen of the inner housing.

A portion of the proximal end of the first lumen of the inner housing may comprise a recessed portion, the recessed portion configured to mate with a corresponding portion of the bung to sealingly engage the bung with the recessed portion of the first lumen of the inner housing. The recessed portion of the proximal end of the first lumen may comprise a region with a substantially constant diameter along the longitudinal axis, and wherein the corresponding portion of the bung comprises a portion having a substantially constant diameter configured to provide an interference fit with the recessed portion of the proximal end of the first lumen. The bung may comprise a circular rim or flange configured to abut the proximal end of the inner housing. The aperture of the bung may comprise ridges running along at least a portion of the length of the bung to engage and provide an interference fit with a cystoscopy tool inserted therethrough.

The proximal end of the outer housing may comprise a flange and the proximal end of the inner housing may additionally or alternatively comprise a flange, wherein the flange of the proximal end of the inner housing may comprise a male portion configured to mate with a corresponding female portion of the flange of the outer housing. The female portion of the flange of the outer housing may additionally or alternatively comprises a rimmed cup extending around the perimeter of the aperture of the proximal end of the outer housing. The male portion of the flange of the inner housing may comprise a rimmed protrusion extending around the perimeter of the aperture of the proximal end of the inner housing for insertion into a corresponding female portion, such as the rimmed cup, of the outer housing.

In another aspect there is provided an insertion tool for inserting an endoscope such as a cystoscope into the body. The tool may therefore be a cystoscopy insertion tool. The tool comprises an inner housing having an aperture at a proximal end and an aperture at a distal end with a first lumen extending therebetween for receiving a cystoscope therethrough and an outer housing having an aperture at a proximal end and an aperture at a distal end having a longitudinal axis extending therebetween and providing a second lumen extending therebetween for receiving the inner housing such that the inner housing is at least partially nested inside the outer housing, the outer housing comprising a first port and a second port, and wherein the first and second ports comprise respective conduits that extend from the outer housing at an acute angle relative to the longitudinal axis and the proximal end. A channel is formed between the inside of the outer housing and the outside of the inner housing when the inner housing is nested in the outer housing, to allow the passage of fluid and/or a biopsy probe to pass from the respective first and second ports and through the channel. The first and second ports are provided on opposing sides of the outer housing.

The first and second ports each comprise a tab providing a female luer lock fitting.

The second lumen of the outer housing may be shaped relative to the dimensions of the inner housing to provide the channel between the inside of the outer housing and the outside of the inner housing.

The insertion tool may further comprise a first sheath coupled to the inner housing, wherein the first sheath is configured to enclose a cystoscope passing through the first lumen, and a second sheath coupled to the outer housing, the second sheath enclosing the first sheath and providing third and fourth lumens on opposing sides of the first sheath for receiving fluid and/or a biopsy tool from the first and second ports respectively via the channel between the inside of the outer housing and the outside of the inner housing.

The second sheath may have an oval cross-section when the cross-section is taken when viewed along the longitudinal axis, wherein each end of the oval provides the respective third and fourth lumens relative to the first sheath which is central to the oval.

A portion of the outer profile of the inner housing may comprise a lipped portion of reduced profile proximate to the distal end of the inner housing for receiving the first sheath. A portion of the outer profile of the outer housing may also comprises a lipped portion of reduced profile proximate to the distal end of the outer housing for receiving the second sheath.

The acute angle at which the respective conduits of the first and second ports of the outer housing extend at relative to the longitudinal axis of the outer housing may be in the range of 25 to 45 degrees, preferably 30 to 35 degrees, preferably 31 degrees.

In some examples the insertion tool further comprises a bung having an aperture, the bung configured to mate with the proximal end of the first lumen of the inner housing, the bung configured to receive and sealingly engage with a cystoscope inserted through the aperture and into the first lumen of the inner housing.

A portion of the proximal end of the first lumen of the inner housing may comprise a recessed portion, the recessed portion configured to mate with a corresponding portion of the bung to sealingly engage the bung with the recessed portion of the first lumen of the inner housing. The recessed portion of the proximal end of the first lumen may comprise a region with a substantially constant diameter along the longitudinal axis, and wherein the corresponding portion of the bung comprises a portion having a substantially constant diameter configured to provide an interference fit with the recessed portion of the proximal end of the first lumen. The bung may comprise a circular rim or flange configured to abut the proximal end of the inner housing. The aperture of the bung may comprise ridges running along at least a portion of the length of the bung to engage and provide an interference fit with a cystoscopy tool inserted therethrough.

The proximal end of the outer housing may comprise a flange and the proximal end of the inner housing may additionally or alternatively comprise a flange, wherein the flange of the proximal end of the inner housing may comprise a male portion configured to mate with a corresponding female portion of the flange of the outer housing. The female portion of the flange of the outer housing may additionally or alternatively comprises a rimmed cup extending around the perimeter of the aperture of the proximal end of the outer housing. The male portion of the flange of the inner housing may comprise a rimmed protrusion extending around the perimeter of the aperture of the proximal end of the inner housing for insertion into a corresponding female portion, such as the rimmed cup, of the outer housing.

In another aspect there is provided a kit of parts for performing an endoscopic procedure, such as a cystoscopy. The kit of parts comprises the insertion tool of any of the previous claims and an endoscope such as a cystoscope.

The kit of parts may further comprise an oblique lens cap for placing over the lens of the cystoscope, wherein the oblique end cap is configure to adjust the field of view of the cystoscopy to be at an angle relative to the longitudinal axis of the cystoscope. The oblique end cap may aid in improving the field of view from the endoscope, for example to view operation of a tool such as a biopsy tool inserted through a lumen parallel to the endoscope.

### DRAWINGS

Embodiments of the disclosure will now be described, by way of example only, with reference to the accompanying drawings, in which:

FIG. 1 shows a perspective view of an example cystoscopy insertion tool;

FIG. 2 shows a perspective view of the example cystoscopy insertion tool of FIG. 1 ready for use;

FIG. 3 shows a perspective view of an example outer housing forming part of a cystoscopy insertion tool such as the example cystoscopy insertion tool shown in FIGS. 1 and 2;

FIG. 4 shows a cross-section of the example outer housing of FIG. 3 taken along its longitudinal axis;

FIG. 5 shows a perspective view of an example inner housing for use with an outer housing, such as the outer housing shown in FIGS. 3 and 4 and forming part of a cystoscopy insertion tool such as that shown in FIGS. 1 and 2;

FIG. 6 shows a cross-section of the example inner housing of FIG. 5 taken along its longitudinal axis;

FIG. 7A shows a perspective view of a bung for use with a cystoscopy insertion tool, such as the tool shown in FIGS. 1 to 6;

FIG. 7B shows a cross-section view of the bung of FIG. 7A taken along its longitudinal axis;

FIG. 8 shows a perspective view of a lens end cap for use with a cystoscopy insertion tool, such as the tool shown in FIGS. 1 to 6;

FIG. 9A shows a perspective view of the distal end of an example cystoscopy insertion tool;

FIG. 9B shows a perspective view of the distal end of the example cystoscopy insertion tool of FIG. 9A with a portion of the inner sheath sealed to a lens end cap; and

FIG. 9C shows a perspective view of the distal end of the example cystoscopy insertion tool of FIGS. 9A and 9B with a portion of the outer sheath sealed to the inner sheath.

### SPECIFIC DESCRIPTION

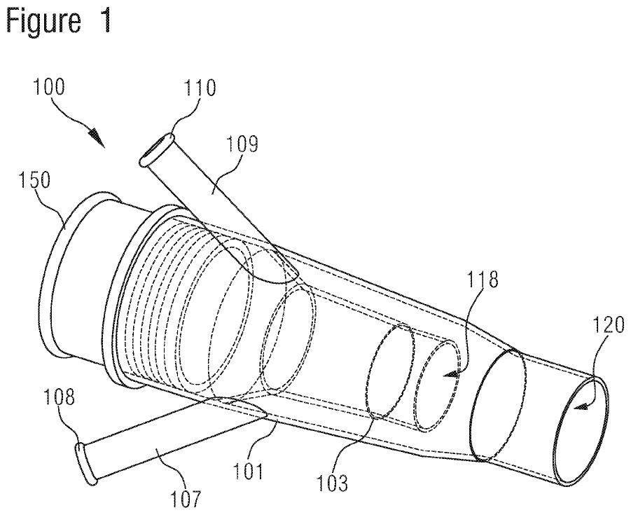

FIG. 1 shows a perspective view of an example cystoscopy insertion tool 100. The cystoscopy insertion tool 100 comprises a tubular inner housing 103, a tubular outer housing 101, and a bung 150. The inner housing 103 is configured to nest within the outer housing 101 and provides a channel therebetween, wherein the boundaries of the channel are formed by the outer profile of the tubular inner housing 103 and the inner profile of the tubular outer housing 101. As shown in FIG. 1, the bung 150 is configured to be inserted into a proximal end of the inner housing 103 and has an aperture 160 for receiving a cystoscope. The outer housing 101 comprises a first port 108 and a second port 110 on an opposite side for receiving fluid and/or a tool such as a biopsy tool, and the ports 108, 110 are coupled via respective conduits 107, 109 to provide access to the channel between the inner housing 103 and the outer housing 101. The channel between the inner housing 103 and the outer housing 101 may therefore act as a common channel to both a tool and fluid delivered from the respective ports/conduits. The insertion tool 100 is configured such that a cystoscope may be inserted through the bung 150 and through the inner housing 103, whereas fluid and/or tools may be delivered through the respective ports 108, 110 in the outer housing 101 and to either side of the cystoscope (e.g. via respective lumens of a sheath) to a site of interest.

The relative dimensions and profiles of the internal surfaces of the bung 150, inner housing 103 and outer housing 101 may be configured to provide a smooth working surface for an endoscope to engage with, to ensure that an endoscope can be inserted smoothly and easily through the insertion tool 100 to a site of interest.

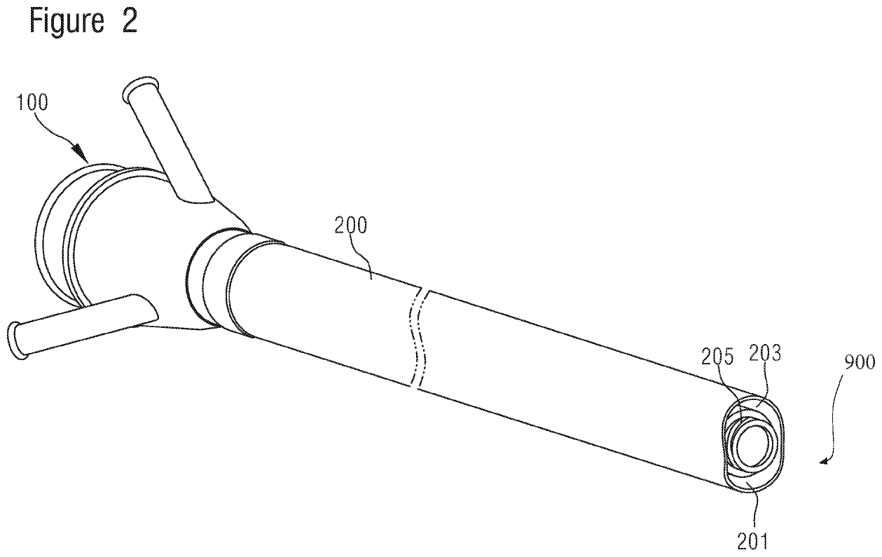

In use, as shown in FIG. 2 which shows a perspective view of the example cystoscopy insertion tool 100 of FIG. 1 ready for use, a cystoscope is inserted through the cystoscopy insertion tool 100. A first sheath 205 is coupled to the inner housing 103 and is configured to enclose a cystoscope passing through the first lumen of the inner housing 103, and a second sheath 200 is coupled to the outer housing 101 enclosing the first sheath 205 and is configured to provide third 201 and fourth 203 lumens on opposing sides of the first sheath 205 for receiving fluid and/or a biopsy tool from the first and second ports 108, 110 respectively via the channel between the inside of the outer housing and the outside of the inner housing. The second sheath 200 may be configured to provide the third 201 and fourth 203 lumens by having a substantially oval or elliptical shape in cross-section perpendicular to its longitudinal axis. In some examples, the second sheath 200 may be adhered to the first sheath 205 to provide the third 201 and fourth 203 lumens. For example, the second sheath 200 may be adhered (e.g., glued and/or heat sealed/welded) to the first sheath 205 axially (i.e., in a direction along the longitudinal axis) to separate regions between the first 205 and second 200 sheaths to thereby form the third 201 and fourth 203 lumens. By providing separate lumens in this manner it may advantageously separate fluid and/or a biopsy tool from each other and advantageously help the clinician guide the delivery of fluid and/or the biopsy tool to the correct location.

The relative orientation of the ports 108, 110, the conduits 107, 109 and the relative dimensions of the inner housing 103 and the outer housing 101 are configured to facilitate the passage of fluid and/or tools through the ports 108, 110 and into the channel between the inner housing and the outer housing 101 and into the respective lumens 201, 203 created by the second sheath 200 either side of the first sheath 205 that encloses the cystoscope in a manner that minimises turbulence and/or friction. In this way, fluid and/or tools may advantageously be effectively delivered to the site of interest. Moreover, fluid may be delivered to the site of interest through one port/conduit at the same time that a tool is being used via another port/conduit. Furthermore the relative orientations of the ports 108, 110 and conduits 107, 109, as well as the geometry of the outer profile of the inner housing 101 that forms one of the boundaries for the channel between the inner housing 101 and outer housing 103 is such that turbulence in the fluid is inhibited, helping to promote a smooth laminar flow that is more comfortable for the patient as it may inhibit the formation of uncomfortable air bubbles. Moreover, the relative orientations of the ports 108, 110 and conduits 107, 109, as well as the geometry of the outer profile of the inner housing 101 are arranged to improve the delivery of a tool through the insertion device, through the second sheath 200 and to the site of interest, by minimising friction to the tool as it is inserted.

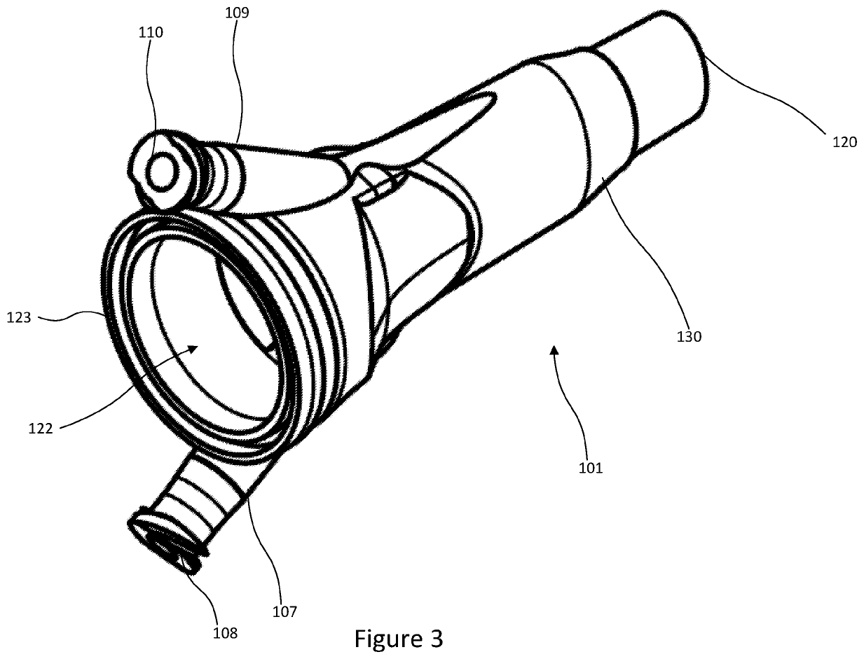

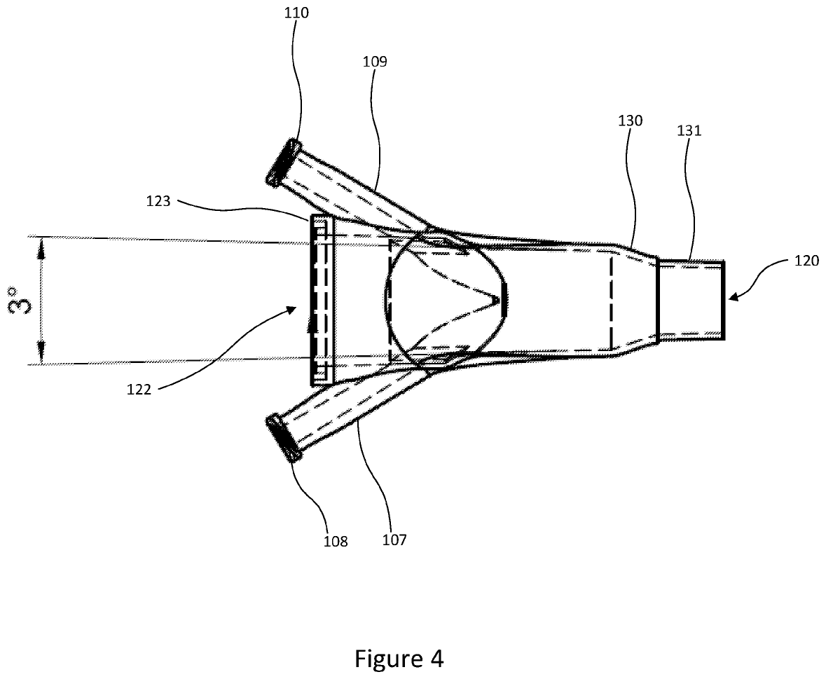

The outer housing 101 is shown in more detail in FIGS. 3 and 4. FIG. 3 shows a perspective view of an example outer housing 101, and FIG. 4 shows a cross-section of the example outer housing 101 taken along its longitudinal axis. As can be seen, the outer housing 101 has an aperture 122 at a proximal end and an aperture 120 at a distal end. The outer housing 101 has a longitudinal axis extending between the aperture 122 at the proximal end and the aperture 120 at the distal end, and is generally tubular such that it provides a second lumen extending therebetween for receiving the inner housing 103 such that the inner housing 103 is at least partially nested inside the outer housing 101. The second lumen of the outer housing 101 is coaxial with the first lumen of the inner housing 103. At the proximal end the outer housing 101 comprises a flange 123 around the rim of the aperture 122. The flange 123 comprises a receiving portion in the form of a circular groove that forms a rimmed cup around the aperture 122. In the example shown the flange 123 extends around the entirety of the circumference of the aperture 122.

The outer housing 103 comprises a first port 108 and a second port 110, and the first and second ports 108, 110 comprise respective conduits 107, 109 that extend from the outer housing 103 at an acute angle relative to the longitudinal axis and the proximal end. The conduits 107, 109 are generally tubular and act as a tube or pipe for conveying tools and/or fluid into the channel between the inner housing 103 and the outer housing 101 from the ports 109, 110. In the example shown, the respective dimensions of the first and second ports 108, 110 and the conduits 107, 109 are identical such that they can be interchanged with each other—in other words a tool may be inserted in one and a fluid through the other and then the tool and fluid may be interchanged.

In the example shown, the acute angle at which the respective conduits 107, 109 of the first and second ports 108, 110 of the outer housing 101 extend at relative to the longitudinal axis of the outer housing 101 is in the range of 25 to 45 degrees, preferably 30 to 35 degrees, preferably 31 degrees. Also, in the example shown, the first and second ports 108, 110 each comprise a tab which in the example shown provides a female luer lock fitting although it will be understood that other types of fitting may be used. This may help in securing a line such as a fluid line to the tool 100 quickly and easily. The second lumen also has a substantially circular cross section and is gently tapered meaning that its cross section narrows from the proximal end to the distal end. In the example shown both the inner profile of the outer housing 101 and the inner profile of the outer housing 101 have the same degree of tapering such that the thickness of material forming the outer housing 101 remains substantially constant along the majority of the length of the outer housing 101 (when travelling along its longitudinal axis), although the thickness of material forming the outer housing 101 is slightly thicker in a region adjacent to/near the proximal end to provide increased strength and rigidity in this region as this region mates with the inner housing 103 and may experience forces applied to it when a cystoscope is inserted through the tool 100.

As can be seen in FIG. 4, the tapering shown in FIG. 4 is about 3 degrees. The outer housing 103 also comprises a frustoconical portion 130, wherein both the outer and inner profiles of the outer housing body 103 narrow to a greater degree, providing a frustoconical cross-section when viewed in cross-section along the longitudinal axis. The frustoconical portion 130 acts to greatly reduce the inner diameter of the outer housing 101 and thereby the diameter of the second lumen such that at the proximal end of the frustoconical portion 130 the second lumen has a first, larger diameter, and at the distal end of the frustoconical portion 130 the second lumen has a second, smaller diameter. The frustoconical portion 130 may aid in inserting the tool 100 at least partially into a body cavity. Adjacent to the frustoconical portion 130 at the distal end of the outer housing 101 is a lipped or stepped portion 131 (shown in FIG. 4). The stepped portion 131 comprises a region with a reduced thickness in the material comprising the outer housing 101 and is configured to receive a sheath (as shown in FIG. 2, it is configured to receive the second sheath 200) such that the sheath provides an interference fit with stepped portion 131 to provide a seal with the outer housing 101. Providing a stepped portion 131 may help to ensure that the sheath such as the second sheath 200 is securely engaged on the outer housing 101.

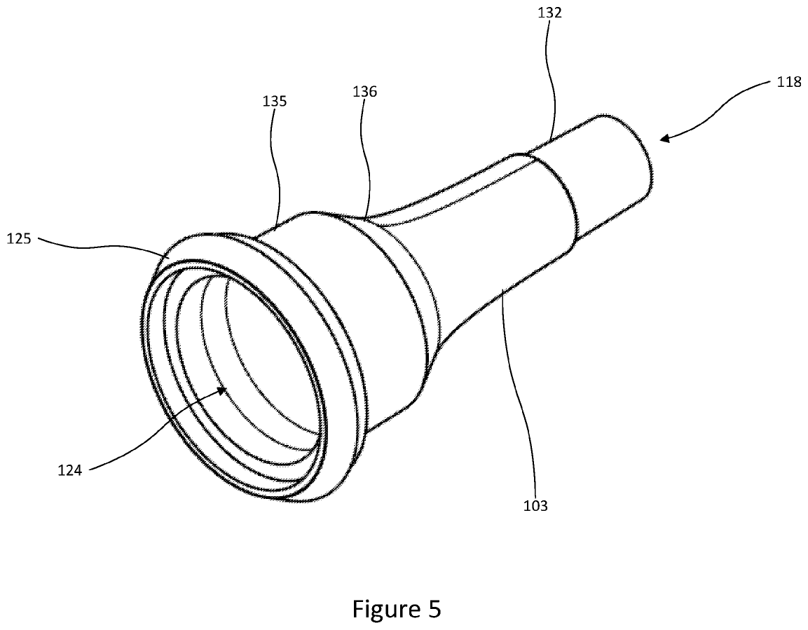

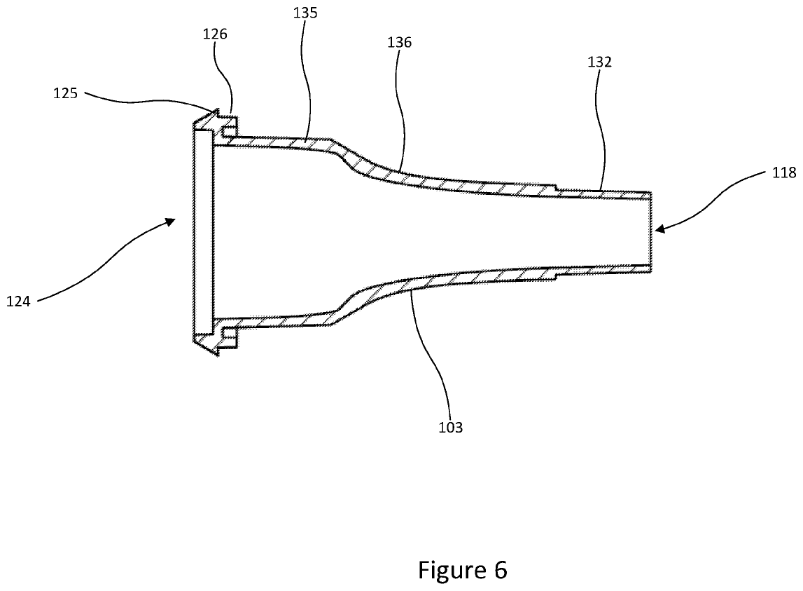

The inner housing 103 is shown in more detail in FIGS. 5 and 6. FIG. 5 shows a perspective view of an example inner housing 103, and FIG. 6 shows a cross-section of the example inner housing 103 taken along its longitudinal axis. The inner housing 103 is generally conical in shape, and has an aperture 124 at a proximal end and an aperture 118 at a distal end with a first lumen extending therebetween for receiving a cystoscope therethrough. The first lumen has a substantially circular cross section which narrows from the proximal end to the distal end.

The inner housing 103 therefore has four main portions. At the proximal end, the inner housing comprises a flange 125 around the rim of the aperture 124. The flange 125 is in the form of a ring around the entirety of the circumference of the aperture 125 and has a protrusion 126 extending therefrom around its circumference. The protrusion 125 is configured to mate with a corresponding recess (for example, the circular groove) of the flange 123 of the outer housing 101 and may, for example, provide an interference fit to inhibit the follow of liquid therebetween. In some examples, during the manufacturing process of the tool 110, the inner housing 103 and the outer housing 101 may be glued together. Providing a mating arrangement in this manner, in particular with a recess in the flange 123 of the outer housing, provides a receptable for adhesive to be captured, ensuring that no adhesive may flow inside the tool and potentially act to inhibit or block the channel, the first lumen and/or second lumen.

Adjacent to the flange 125 at the proximal end, the inner housing 103 comprises a substantially cylindrical portion 135 that has little or no taper along the length of the longitudinal axis which corresponds to and is coaxial with the first lumen. As a result, the substantially cylindrical portion 135 has a substantially constant interior diameter along the longitudinal axis of the inner housing 103. This cylindrical portion 135 is configured to receive the bung 150.

Adjacent to the cylindrical portion is a tapered portion 136. In the example shown, the tapered portion 136 is arranged such that both the inner and outer profiles of this portion taper to approximately the same degree along the longitudinal axis, such that the thickness material forming the body of the inner housing 103 remains substantially constant. However, it will be understood that in other examples the thickness of the material may vary and it may only be the outer profile of the inner housing 103 that tapers. The degree of tapering of the external profile of the tapered portion 136 of the inner housing 103 is based on the angle at which the respective conduits of the first and second ports of the outer housing extend at relative to the longitudinal axis of the outer housing. In the example shown, it can be seen that the tapered portion 136 has a generally arcuate profile in cross-section, with the steepest angle of taper being proximate to the proximal end and the shallowest angle of taper being proximate to the distal end. The arcuate profile may be based on an exponential function.

The arcuate profile is arranged to provide a smooth interface for fluid and/or tools flowing through the conduits 107, 109 of the outer housing 101 to come against, thereby guiding or funnelling fluid and/or tools through the conduits 107, 109 and into the channel formed between the external profile of the inner housing 103 and the interior profile of the outer housing 101.

At the distal end of the inner housing 103 there is a lipped or stepped portion 132. As with the stepped portion 131 of the outer housing, the stepped portion 132 comprises a region with a reduced thickness in the material comprising the outer housing 101 and is configured to receive a sheath (as shown in FIG. 2, it is configured to receive the first sheath 205) such that the sheath provides an interference fit with stepped portion 132 to provide a seal with the inner housing 103. Providing a stepped portion 132 may help to ensure that the sheath such as the first sheath 205 is securely engaged on the inner housing 103.

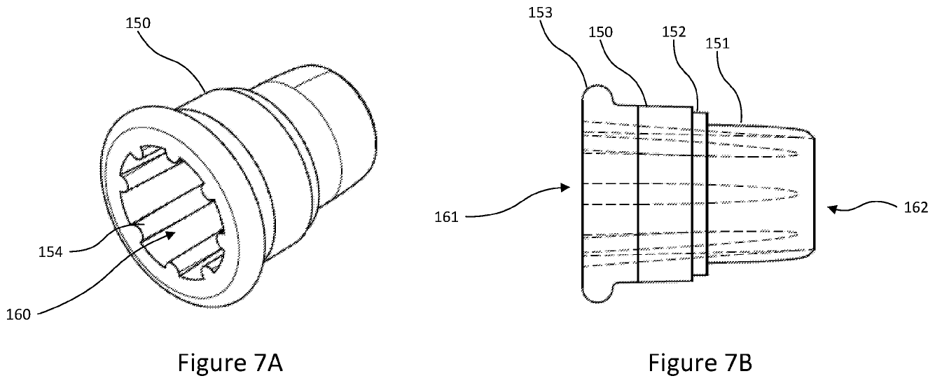

FIG. 7A shows a perspective view of a bung 150 for use with a cystoscopy insertion tool, such as the tool shown in FIGS. 1 to 6 and FIG. 7B shows a cross-section view of the bung of FIG. 7A taken along its longitudinal axis. As noted above, the bung 150 is configured to be inserted into the proximal end of the inner housing 103 and has a bung lumen 160 extending therethrough for receiving a cystoscope. The bung 150 is therefore configured to act as a flexible interface between the relatively rigid cystoscope and the relatively rigid inner and outer housings 103, 101.

In more detail, the bung 150 has a proximal end 161 and a distal end 162, with a bung lumen 160 extending therethrough that is coaxial with the longitudinal axis of the bung 150. The distal end 162 has a narrowed mating portion 151 that is configured to be inserted into the first lumen of the inner housing 103, and specifically be received by the substantially cylindrical portion 135, and as such the mating portion 151 has an exterior profile that matches that of the inner profile of the cylindrical portion 135. The mating portion 151 of the bung 150 is configured to mate with the substantially cylindrical portion 135 of the first lumen of the inner housing 103 and to provide an interference fit with the substantially cylindrical portion 135 and the interior surface of the first lumen of the inner housing 103 to secure and sealingly engage the bung 150 in the first lumen of the inner housing 103. To this end, the distal end 162 of the bung has a chamfered edge to aid insertion into the first lumen of the inner housing 103.

Although the mating portion 151 of the bung 150 and the cylindrical portion 135 of the inner housing 103 are described above as having substantially constant diameters along the longitudinal axis, it will be appreciated that in some examples they both might have a slight taper, for example of around 3 degrees, which may aid insertion and improve the interference fit between the bung 150 and the inner housing 103.

Adjacent to the mating portion 151 on the proximal side is a stepped portion 152. The stepped portion 152 has an increased outer diameter relative to the mating portion 151 that acts as a flange and is configured to engage with and abut the proximal end of the inner housing 103 and also the proximal end of the outer housing 101. In the example the stepped portion 152 comprises two steps, one to abut the proximal end of the inner housing 103 and one to abut the proximal end of the outer housing 103.

Adjacent to the stepped portion 152 is a tapered portion leading to a circular rim 153 at the proximal end of the bung 150. The circular rim 153 has an external profile with an increased diameter relative to the rest of the bung 150 and is configured to be gripped by a user to facilitate insertion and extraction of the bung 150 from the inner housing 103.

The bung lumen 160 is slightly tapered from the proximal end 161 to the distal end 162 and has a plurality of ridges 154 extending along at least a portion of the bung lumen 160. In the example shown, the ridges 154 extend almost entirely along the entire length of the bung lumen 160. The ridges 154 are evenly spaced around the interior surface of the bung lumen 160 and provide a series of peaks and troughs for engaging with a cystoscope inserted in the bung lumen 160. In the example shown the ridges 154 have rounded or chamfered peaks to provide a rounded interface for the cystoscope to engage, but it will be understood that in other examples the ridges 154 may have other profiles. The ridges 154 may act to improve engagement of a cystoscope inserted through the bung lumen 160.

It will be understood that the cystoscopy insertion tool 100, comprising the inner housing 103, the outer housing 101 and the bung 150 may be provided as a kit of parts, for example as part of a giving set or a sealed sterile pack. In some examples, the cystoscopy insertion tool 100 may be provided as a kit of parts comprising a cystoscope.

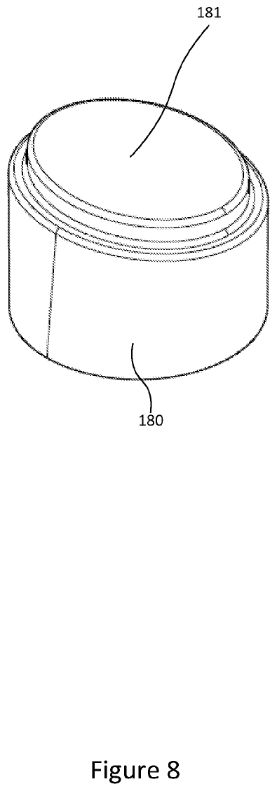

In some examples, the cystoscopy insertion tool 100 may further comprise a lens cap 180 as shown in FIG. 8 at the distal end 900 of the cystoscopy insertion tool 100. The lens cap 180 may be configured to be secured to the distal end of the first sheath 205 described above with reference to FIG. 2. For example, the lens cap 180 may be inserted into the distal end of the first sheath 205 and secured by an interference fit and/or with adhesive or other means such as a weld, for example a heat weld. The lens cap 180 may have an oblique end face 181. The oblique end face 181 is configured to adjust the field of view of the cystoscopy to be at an angle relative to the longitudinal axis of the cystoscope. This may aid in improving the field of view of, for example, a biopsy tool inserted through the third or fourth lumens 201, 203.

Additionally, or alternatively to the lens cap 180 having an oblique end face 181, the lens cap 180 may be generally oval shaped. Advantageously, an oval shaped lens cap 180 may alter the shape, for example, of the third 201 and/or fourth 203 lumens and help bring any tool such as a biopsy tool to emerge from the end of the third 201 and/or fourth 203 lumens and/or the gap between the first sheath 205 and the second sheath 200 at the correction location relative to the lens cap 180, which in turn may make use of the tool easier for a clinician.

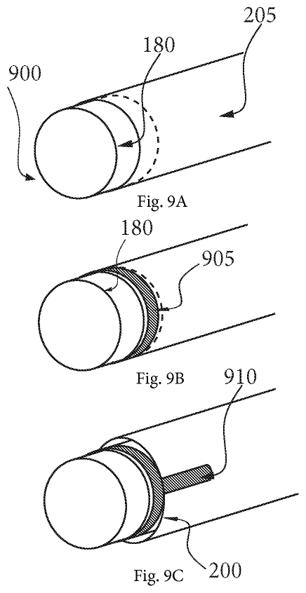

The lens cap 180 may be glued to the to the end of the first sheath 205. To avoid the inefficiencies and high scrap rates of manual assembly and gluing, another embodiment as shown in FIGS. 9A and 9B may use heat sealing to bond the lens cap 180 to the first sheath 205. In this manner, the lens cap 180 may be placed on an inner rod which is inserted into the first sheath 205 until it protrudes a defined distance from the end. The area of overlap 905 is placed on a ‘pillow’ block which allows heat to be applied around the collar creating an annular seal. The block typically covers 180 degrees, so the assembly is rotated and the procedure repeated to give a good 360 degree seal. For example, the assembly may be rotated 120 degrees, heat sealed, then rotated another 120 degrees, then heat sealed, and finally rotated once more 120 degrees and then heat sealed, to provide a good 360 degree seal.

The second sheath 200 must also be attached to the assembly (so that the second sheath 200 does not roll back when the device is inserted), but the attaching points preferably leave room for irrigation and biopsy tool egress at the distal end 900. As shown in FIGS. 9A to 9C, a proposal is for the above heat sealing to include a small axial portion 910 at two points which weld the second sheath 200 to the first sheath 205/lens assembly 180. This can be done at the same time, saving assembly costs. The axial sealing pattern may run down the full length of the first 205 and second 200 sheaths, or just a partial length. One clinical benefit of the axial sealing is that the biopsy tool will emerge from a relatively fixed point at the distal end 900, and not be prone to moving circumferentially during operation. Of course, although only a small axial portion 910 of a seal is shown in FIG. 9C, in some examples the axial portion 910 of the seal may extend further in the longitudinal axis. However, advantageously a small axial portion 910 of a seal means that the seal can be created at the same time as welding/adhering the lens cap 180 to the first sheath 205.

Additionally, or alternatively, to further aid in guiding a tool such as a biopsy tool through the third 201 and/or fourth lumen 203 such that it exits the distal end 900 of the cystoscopy insertion tool 100 at the correct location, the second sheath 200 may comprise one or more cut-outs, such as a hole, at or proximate to the distal end 900 and proximate to the lens cap 180. The remaining distal portion of the sheath 200 beyond the hole (in a distal direction) may preferably be bonded in a complete annular/circumferential weld. Providing such a cut-out or hole proximate to the distal end 900 has surprisingly been found to preferentially guide a tool such as a biopsy tool from the location of the cut-out. Advantageously this means that a clinician when using the cystoscopy insertion tool 100 can guide the biopsy tool to the correct location relative to the lens cap 180 and thereby relative to their field of view as being viewed through the camera.

Although the third and fourth lumens 201, 203 shown in FIG. 2 appear to be the same size and on opposing sides of the first sheath 205, in some examples the third and fourth lumens 201, 203 may be of differing sizes. For example, in some examples the third lumen 201 may be smaller (in terms of cross-sectional area and/or volume) than the fourth lumen 203. In some examples, the third 201 and fourth 203 lumens may be separated by a pair of seals (e.g., glue or weld) in an axial direction (i.e., along the longitudinal axis) separated by approximately 90 degrees (in a plane perpendicular to the longitudinal axis). In this way, the volume of the fourth lumen 203 may be, for example, at least three times greater than the volume of the third lumen 201.

It will be appreciated from the discussion above that the embodiments shown in the Figures are merely exemplary, and include features which may be generalised, removed or replaced as described herein and as set out in the claims.

It will be understood that the cystoscopy insertion tool 100, including the inner housing 103, the outer housing 101 and/or the bung 150, may be manufactured by subtractive or additive processes. For example, the inner housing 103, the outer housing 101 and/or the bung 150 may be manufactured using 3D printing using a PLA thermoplastic material.

The inner housing 103, the outer housing 101 and/or the bung 150, may also be manufactured by assembling pre-manufactured components together such as by adhering a sheetlike element to a substrate. This may be done by laying down a preformed track of the material, or by laying down a larger sheet and then etching it away. This sheetlike element may be grown or deposited as a layer on the substrate. If it is deposited a mask may be used so the deposition happens only on regions which are to carry the track and/or it may be allowed to take place over a larger area and then selectively etched away.

Other methods of manufacture may also be used. For example, the inner housing 103, the outer housing 101 and/or the bung 150 may be manufactured by way of ‘3D printing’ whereby a three-dimensional model of the inner housing 103, the outer housing 101 and/or the bung 150 are supplied, in machine readable form, to a ‘3D printer’ adapted to manufacture the inner housing 103, the outer housing 101 and/or the bung 150. This may be by additive means such as extrusion deposition, Electron Beam Freeform Fabrication (EBF), granular materials binding, lamination, photopolymerization, or stereolithography or a combination thereof. The machine readable model comprises a spatial map of the object to be printed, typically in the form of a Cartesian coordinate system defining the object's surfaces. This spatial map may comprise a computer file which may be provided in any one of a number of file conventions. One example of a file convention is a STL (STereoLithography) file which may be in the form of ASCII (American Standard Code for Information Interchange) or binary and specifies areas by way of triangulated surfaces with defined normals and vertices. An alternative file format is AMF (Additive Manufacturing File) which provides the facility to specify the material and texture of each surface as well as allowing for curved triangulated surfaces. The mapping of the inner housing 103, the outer housing 101 and/or the bung 150 may then be converted into instructions to be executed by 3D printer according to the printing method being used. This may comprise splitting the model into slices (for example, each slice corresponding to an x-y plane, with successive layers building the z dimension) and encoding each slice into a series of instructions. The instructions sent to the 3D printer may comprise Numerical Control (NC) or Computer NC (CNC) instructions, preferably in the form of G-code (also called RS-274), which comprises a series of instructions regarding how the 3D printer should act. The instructions vary depending on the type of 3D printer being used, but in the example of a moving printhead the instructions include: how the printhead should move, when/where to deposit material, the type of material to be deposited, and the flow rate of the deposited material.

Although the tool has been described as a cystoscopy insertion tool for inserting a cystoscope into the body, it will be appreciated that the tool may be adapted and used for inserting an instrument, such as an endoscopy, in other body cavities. For example, the tool could be adapted for use in performing a bronchoscopy or a sigmoidoscopy.

Although the second sheath 200 is described as providing third and fourth lumens 201, 203, in some examples the second sheath 200 may be configured to provide additional lumens—for example, for enclosing a bundle of optical fibres for performing laser treatment and ablation at a treatment site.

In the context of the present disclosure other examples and variations of the apparatus and methods described herein will be apparent to a person of skill in the art.

1. A cystoscopy insertion tool for inserting a cystoscope into the body, the tool comprising:
an inner housing having an aperture at a proximal end and an aperture at a distal end with a first lumen extending therebetween for receiving a cystoscope therethrough;
an outer housing having an aperture at a proximal end and an aperture at a distal end having a longitudinal axis extending therebetween and providing a second lumen extending therebetween for receiving the inner housing such that the inner housing is at least partially nested inside the outer housing, the outer housing comprising a first port and a second port, and wherein the first and second ports comprise respective conduits that extend from the outer housing at an angle relative to the longitudinal axis and the proximal end;
a channel between the inside of the outer housing and the outside of the inner housing when the inner housing is nested in the outer housing, to allow the passage of fluid and/or a biopsy probe to pass from the respective first and second ports and through the channel; and
wherein at least a portion of the external profile of the inner housing is tapered, wherein the degree of tapering is based on the angle at which the respective conduits of the first and second ports of the outer housing extend at relative to the longitudinal axis of the outer housing.

2.-24. (canceled)

25. The cystoscopy insertion tool of claim 1 wherein the tapering of the external profile of the inner housing is at least partially arcuate.

26. The cystoscopy insertion tool of claim 1 wherein the at least a portion of the external profile of the inner housing is tapered along the length of the first region at a region proximate to the respective conduits of the first and second ports when the inner housing is nested inside the outer housing.

27. The cystoscopy insertion tool of claim 1 wherein the tapering of the external diameter of the inner housing and the relative angle at which the respective conduits of the first and second ports of the outer housing extend at relative to the longitudinal axis of the outer housing is selected to provide a laminar flow of fluid through the respective conduits and through the channel between the inside of the outer housing and the outside of the inner housing.

28. The cystoscopy insertion tool of claim 1 wherein the outer housing is tapered at least partially along its longitudinal axis.

29. The cystoscopy insertion tool of claim 1 wherein the first and second ports are (i) provided on opposing sides of the outer housing, and/or (ii) each comprise a tab providing a female luer lock fitting.

30. The cystoscopy insertion tool of claim 1 wherein the second lumen of the outer housing is shaped relative to the dimensions of the inner housing to provide the channel between the inside of the outer housing and the outside of the inner housing.

31. The cystoscopy insertion tool of claim 1 comprising:
a first sheath coupled to the inner housing, wherein the first sheath is configured to enclose a cystoscope passing through the first lumen;
a second sheath coupled to the outer housing, the second sheath enclosing the first sheath and providing third and fourth lumens on opposing sides of the first sheath for receiving fluid and/or a biopsy tool from the first and second ports respectively via the channel between the inside of the outer housing and the outside of the inner housing.

32. The cystoscopy insertion tool of claim 31 wherein the second sheath has an oval cross-section when the cross-section is taken relative to the longitudinal axis, wherein each end of the oval provides the respective third and fourth lumens relative to the first sheath which is central to the oval, and,
wherein a portion of the outer profile of the inner housing comprises a lipped portion of reduced profile proximate to the distal end of the inner housing for receiving the first sheath, and wherein a portion of the outer profile of the outer housing comprises a lipped portion of reduced profile proximate to the distal end of the outer housing for receiving the second sheath.

33. The cystoscopy insertion tool of claim 1 further comprising a bung having an aperture, the bung configured to mate with the proximal end of the first lumen of the inner housing, the bung configured to receive and sealingly engage with a cystoscope inserted through the aperture and into the first lumen of the inner housing,
wherein a portion of the proximal end of the first lumen of the inner housing comprises a recessed portion, the recessed portion configured to mate with a corresponding portion of the bung to sealingly engage the bung with the recessed portion of the first lumen of the inner housing and wherein the recessed portion of the proximal end of the first lumen comprises a region with a substantially constant diameter along the longitudinal axis, and
wherein the corresponding portion of the bung comprises a portion having a substantially constant diameter configured to provide an interference fit with the recessed portion of the proximal end of the first lumen;
wherein the bung comprises a flange configured to abut the proximal end of the inner housing; and
wherein the aperture of the bung comprises ridges running along at least a portion of the length of the bung to engage and provide an interference fit with a cystoscopy tool inserted therethrough.

34. A kit of parts for performing cystoscopy, comprising:
the cystoscopy insertion tool of claim 1; and
a cystoscope.

35. A cystoscopy insertion tool for inserting a cystoscope into the body, the tool comprising:
an inner housing having an aperture at a proximal end and an aperture at a distal end with a first lumen extending therebetween for receiving a cystoscope therethrough;
an outer housing having an aperture at a proximal end and an aperture at a distal end having a longitudinal axis extending therebetween and providing a second lumen extending therebetween for receiving the inner housing such that the inner housing is at least partially nested inside the outer housing, the outer housing comprising a first port and a second port, and wherein the first and second ports comprise respective conduits that extend from the outer housing at an acute angle relative to the longitudinal axis and the proximal end;
a channel between the inside of the outer housing and the outside of the inner housing when the inner housing is nested in the outer housing, to allow the passage of fluid and/or a biopsy probe to pass from the respective first and second ports and through the channel; and
wherein the first and second ports are provided on opposing sides of the outer housing.

36. The cystoscopy insertion tool of claim 35 wherein the second lumen of the outer housing is shaped relative to the dimensions of the inner housing to provide the channel between the inside of the outer housing and the outside of the inner housing.

37. The cystoscopy insertion tool of claim 35 comprising:
a first sheath coupled to the inner housing, wherein the first sheath is configured to enclose a cystoscope passing through the first lumen;
a second sheath coupled to the outer housing, the second sheath enclosing the first sheath and providing third and fourth lumens on opposing sides of the first sheath for receiving fluid and/or a biopsy tool from the first and second ports respectively via the channel between the inside of the outer housing and the outside of the inner housing.

38. The cystoscopy insertion tool of claim 37 wherein the second sheath has an oval cross-section when the cross-section is taken relative to the longitudinal axis, wherein each end of the oval provides the respective third and fourth lumens relative to the first sheath which is central to the oval, and,
wherein a portion of the outer profile of the inner housing comprises a lipped portion of reduced profile proximate to the distal end of the inner housing for receiving the first sheath, and wherein a portion of the outer profile of the outer housing comprises a lipped portion of reduced profile proximate to the distal end of the outer housing for receiving the second sheath.

39. The cystoscopy insertion tool of claim 35 wherein the acute angle at which the respective conduits of the first and second ports of the outer housing extend at relative to the longitudinal axis of the outer housing is in the range of 25 to 45 degrees, preferably 30 to 35 degrees, preferably 31 degrees.

40. The cystoscopy insertion tool of claim 35 further comprising a bung having an aperture, the bung configured to mate with the proximal end of the first lumen of the inner housing, the bung configured to receive and sealingly engage with a cystoscope inserted through the aperture and into the first lumen of the inner housing;
wherein a portion of the proximal end of the first lumen of the inner housing comprises a recessed portion, the recessed portion configured to mate with a corresponding portion of the bung to sealingly engage the bung with the recessed portion of the first lumen of the inner housing;
wherein the recessed portion of the proximal end of the first lumen comprises a region with a substantially constant diameter along the longitudinal axis;
wherein the corresponding portion of the bung comprises a portion having a substantially constant diameter configured to provide an interference fit with the recessed portion of the proximal end of the first lumen;
wherein the bung comprises a flange configured to abut the proximal end of the inner housing; and
wherein the aperture of the bung comprises ridges running along at least a portion of the length of the bung to engage and provide an interference fit with a cystoscopy tool inserted therethrough.

41. The cystoscopy insertion tool of claim 35 wherein the proximal end of the outer housing comprises a flange and the proximal end of the inner housing comprises a flange;
wherein the flange of the proximal end of the inner housing comprises a male portion configured to mate with a corresponding female portion of the flange of the outer housing;
wherein the female portion of the flange of the outer housing comprises a rimmed cup extending around the perimeter of the aperture of the proximal end of the outer housing; and
wherein the male portion of the flange of the inner housing comprises a rimmed protrusion extending around the perimeter of the aperture of the proximal end of the inner housing.

42. A kit of parts for performing cystoscopy, comprising:
the cystoscopy insertion tool of claim 35; and
a cystoscope.

43. The kit of parts of claim 42, further comprising an oblique lens cap for placing over the lens of the cystoscope, wherein the oblique end cap is configured to adjust the field of view of the cystoscopy to be at an angle relative to the longitudinal axis of the cystoscope.
